(19) United States
(12) Patent Application Publication
(10) Pub. No.: US 2005/0187403 A1
(43) Pub. Date: Aug. 25, 2005

(54) Process for producing 1,3-naphthalenedicarboxylic acid

(76) Inventors: Hiroshi Ogawa, Okayama (JP); Junya Nishiuchi, Okayama (JP); Mitsuharu Kitamura, Okayama (JP); Kinji Kato, Okayama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/063,970

(22) Filed: Feb. 24, 2005

(30) Foreign Application Priority Data
Feb. 25, 2004 (JP) 2004-49025
Feb. 25, 2004 (JP) 2004-49027

(51) Int. Cl. C07C 51/255

(52) U.S. Cl. 562/416

(57) ABSTRACT

1,3-Naphthalenedicarboxylic acid is produced by oxidizing 1,3-dialkylnaphthalene in a liquid-phase with an oxygen-containing gas in the presence of a C₂-C₆ lower aliphatic carboxylic acid solvent and a catalyst comprising a heavy metal and a bromine compound. By regulating the ratio of the total number of bromine atoms fed into a reaction system to the total number of 1,3-dialkylnaphthalene molecules fed into the reaction system within a specific range, 1,3-naphthalenedicarboxylic acid is efficiently produced with low costs. Using 1,3-dimethylnaphthalene, as the starting 1,3-dialkylnaphthalene, which is produced by isomerizing dimethylnaphthalenes in a liquid phase in the presence of a catalyst comprising hydrogen fluoride and boron trifluoride together with a C₅-C₁₀ alicyclic saturated hydrocarbon having a five-membered or six-membered ring structure, a highly pure 1,3-naphthalenedicarboxylic acid is efficiently produced.

### BACKGROUND OF THE INVENTION

1. Field of the Invention

The present invention relates to a process for producing 1,3-naphthalenedicarboxylic acid. Hereinafter, naphthalenedicarboxylic acid is referred to as “NDCA.” 1,3-NDCA and esters thereof are extensively used in wide applications such as raw materials for polyester resins and fibers having unprecedentedly useful functions, raw materials for liquid crystal polymers, modifiers for polyesters, curing agents for epoxy resins, raw materials for medicines or agricultural chemicals, and raw materials for lubricants. The present invention further relates to a process for producing 1,3-dimethylnaphthalene useful as a raw material for the production of 1,3-NDCA. Hereinafter, dimethylnaphthalene is referred to as “DMN.”

2. Description of the Prior Arts

It is known that 1,3-NDCA is produced by oxidizing 1,3-DMN with an oxidizing agent such as Na₂Cr₂O₇ (Bull. Chem. Soc. Jpn., 62, 3, 1989, 786-790). It is also known that 2,6-NDCA, an isomer of 1,3-NDCA, is produced by oxidizing 2,6-DMN with a molecular oxygen in a solvent such as acetic acid in the presence of a catalyst such as Co, Mn and bromine (Japanese Patent No. 3390169, JP 10-316615A and JP 2002-510287A).

However, none of these conventional methods are fully satisfactory for industrial use. For example, in the methods using Na₂Cr₂O₇, etc. as the oxidizing agent, the oxidizing agent should be used in an equivalent amount or more to 1,3-DMN, resulting in a poor production economy. In addition, after the reaction, heavy metals which are extremely harmful to the environment are by-produced from the oxidizing agent used, this considerably increasing the costs for treating the heavy metals. In the method in which 1,3-dialkylnaphthalene is oxidized with molecular oxygen in a solvent such as acetic acid in the presence of a catalyst such as Co, Mn and bromine, the naphthalene ring is likely to be opened during the oxidation reaction to cause the combustion or the conversion into phthalic acid, etc., making it difficult to produce the aimed 1,3-NDCA in high yields. Therefore, it has been demanded to establish an industrially efficient method for producing 1,3-NDCA at low costs.

To eliminate the purification step and to produce a high purity 1,3-NDCA, it is preferable to use a highly pure 1,3-dialkylnaphthalene as the starting compound for its production. The dialkylnaphthalenes, in particular, DMNs which are preferred for industrial use include ten kinds of isomers according to the positions of two methyl groups. It is highly advantageous for the efficient production of a high purity 1,3-NDCA if 1,3-DMN containing substantially no isomers other than 1,3-DMN can be produced from the isomers in a large amount at low costs.

With respect to the isomerization of DMN, it is known that, as compared with the 1,2-shift of methyl group (shift of methyl group between 2-position and 3-position of naphthalene ring), the 2,3-shift and the shift from one ring to the other are difficult to occur. Therefore, the DMN isomers are classified into the following four isomerization groups.

Group A: 1,5-isomer, 1,6-isomer and 2,6-isomer

Group B: 1,8-isomer, 1,7-isomer and 2,7-isomer

Group C: 1,4-isomer, 1,3-isomer and 2,3-isomer

Group D: 1,2-isomer

The isomerization between different groups hardly occurs as compared to the isomerization within the same group.

As the method for the production of 1,3-DMN, there have been known a method of methylating naphthalene or methylnaphthalene, a method of separating 1,3-DMN from tar fractions or petroleum fractions, etc. However, these conventional methods require the separation of 1,3-DMN from the other isomers to fail to provide an effective process for the production of 1,3-DMN.

There has been proposed a method for producing 5-phenylhexene-2 from ethylbenzene and butadiene in a high yield (JP 49-134634A and U.S. Pat. No. 3,244,758). In addition, there has been proposed a method of producing 1,4-DMN by cyclizing 5-phenylhexene-2 using an acid solid catalyst into 1,4-dimethyltetralin which is then dehydrogenated into 1,4-DMN (U.S. Pat. No. 3,775,497). Since 1,4-DMN and 1,3-DMN belong to the same isomerization group, it is advantageous to use 1,4-DMN produced by the proposed method as the starting compound for the production of 1,3-DMN because 1,4-DMN is converted into 1,3-DMN without via the difficult isomerization between the different isomerization groups. As the method of producing 1,3-DMN by the isomerization of 1,4-DMN, U.S. Pat. No. 3,109,036 discloses a method of isomerizing 1,4-DMN into 1,3-DMN in liquid phase in the presence of a catalyst comprising hydrogen fluoride (hereinafter referred to as “HF”) and boron trifluoride (hereinafter referred to as “BF₃”). Although the sole use of HF-BF₃ as the catalyst allows the isomerization of 1,4-DMN into 1,3-DMN in a high isomer selection, the isomerization should be conducted at a relatively high temperature of 0 to 100° C. using a large amount of the HF-BF₃ catalyst to likely cause a side reaction such as the decomposition or polymerization of DMN. To prevent such a side reaction, the use of a large amount of solvent is required to reduce the volume efficiency of the reaction apparatus. Although the side reaction is prevented by conducting the isomerization at a low temperature, the isomerization is not completed within a short period of time to leave a large amount of 1,4-DMN not isomerized.

### SUMMARY OF THE INVENTION

A first object of the present invention is to provide a process for efficiently producing 1,3-NDCA at low costs. A second object of the present invention is to solve the problems in the known isomerization into 1,3-DMN using the HF-BF₃ catalyst mentioned above and provide a process for producing 1,3-DMN with a high production efficiency.

As a result of extensive research in view of achieving the objects, the inventors have found that the opening of the naphthalene ring is reduced and the yield of 1,3-NDCA is increased by oxidizing 1,3-dialkylnaphthalene with an inexpensive oxygen-containing gas in the presence of a catalyst comprising heavy metal and bromine while controlling the ratio of the number of bromine atoms to the number of 1,3-dialkylnaphthalene molecules which are fed into the reaction system within a specific range. It has been further found that 1,4-DMN and 2,3-DMN are isomerized into 1,3-DMN in high isomer selections even at low temperatures at which side reactions such as the decomposition or polymerization of DMN are prevented when the isomerization is conducted in the presence of the HF-BF₃ catalyst together with an alicyclic saturated hydrocarbon having a five-membered or six-membered ring structure in a small amount to DMN. The present invention has been accomplished on the basis of these findings.

Thus, the present invention relates to a process for producing 1,3-NDCA comprising a step of subjecting 1,3-dialkylnaphthalene to a liquid-phase oxidation with an oxygen-containing gas in the presence of a C₂-C₆ lower aliphatic carboxylic acid solvent and a catalyst comprising a heavy metal and a bromine compound, wherein the ratio of the total number of bromine atoms fed into a reaction system to the total number of 1,3-dialkylnaphthalene molecules fed into the reaction system is regulated within the range of 0.015 to 0.30.

The present invention still further relates to the process for producing 1,3-NDCA wherein the starting 1,3-dialkylnaphthalene is a high purity 1,3-DMN which is produced by isomerizing DMN in a liquid phase in the presence of a catalyst comprising HF and BF₃ together with a C₅-C₁₀ alicyclic saturated hydrocarbon having a five-membered or six-membered ring structure.

The present invention further relates to 1,3-NDCA produced by the above production method and 1,3-naphthalenedicarboxylic diester produced by esterifying the 1,3-NDCA.

### DETAILED DESCRIPTION OF THE INVENTION

Examples of the starting 1,3-dialkylnaphthalenes for the production of 1,3-NDCA include 1,3-dimethylnaphthalene, 1,3-diethylnaphthalene, 1,3-diisopropylnaphthalene, 1-methyl-3-ethylnaphthalene, 1-methyl-3-isopropylnaphthalene, 1-ethyl-3-methylnaphthalene and 1-isopropyl-3-methylnaphthalene. Also usable are compounds, exclusive of 1,3-NDCA, derived from the oxidation of one or two alkyl groups of 1,3-dialkylnaphthalenes, such as 1-methyl-3-acetylnaphthalene, 1-methyl-3-formylnaphthalene, 3-methylnaphthoic acid, 1-acetyl-3-methylnaphthalene and 1-formyl-3-methylnaphthalene. Of these compounds, most preferred for industrial use is 1,3-dimethylnaphthalene. Using DMN containing 97% by weight or higher of 1,3-isomer which is produced by the isomerization in the presence of HF-BF₃, 1,3-NDCA with a purity as high as 99% by weight or higher can be produced in the process of the invention.

The lower aliphatic carboxylic acid used as a solvent for the production of 1,3-NDCA is a C₂-C₆ aliphatic monocarboxylic acid such as acetic acid, propionic acid, butyric acid and mixtures thereof, with acetic acid and propionic acid being preferred and acetic acid being particularly preferred. The water content of the solvent is preferably 50% by weight or less. If the reuse of the solvent is intended, the water content is preferably 2 to 50% by weight and more preferably 5 to 30% by weight. When the mother liquor containing the water generated during the reaction is reused in the next liquid-phase oxidation, a complicated dehydration step is needed to reduce the water content if the water content of the solvent is set to a level far smaller than 2% by weight, thereby reducing the production efficiency. If the water content is too high, the reaction rate is lowered to reduce the yield. The amount of the solvent to be used is preferably 2 to 30 times and more preferably 3 to 25 times the 1,3-dialkylnaphthalene by weight.

The catalyst for use in the production of 1,3-NDCA is a combination of at least one heavy metal selected from the group consisting of cobalt, manganese, nickel, cerium, iron and zirconium with a bromine compound, preferably a combination of at least one heavy metal selected from the group consisting of cobalt, manganese and zirconium with the bromine compound, and more preferably a combination of at least one heavy metal selected from the group consisting of cobalt and manganese with the bromine compound. Examples of the heavy metal sources include metal compounds such as organic acid salts, halides and carbonates of the heavy metals, with acetic acid salts and bromides being preferred. The bromine compound is not particularly limited as long as the compound is capable of generating bromine ions in the reaction system. Examples of the bromine compounds include inorganic bromides such as hydrogen bromide, sodium bromide and manganese bromide, and organic bromides such as tetrabromoethane, with hydrogen bromide, cobalt bromide and manganese bromide being preferred. The heavy metal source also serves as the bromine compound if it is a bromide.

The ratio of the total number of bromine atoms fed into the reaction system to the total number of 1,3-dialkylnaphthalene molecules fed into the reaction system (total bromine/total dialkylnaphthalene) is 0.015 to 0.30 and preferably 0.015 to 0.15. If the ratio is less than 0.015, the ring opening reaction of naphthalene ring becomes dominant to extremely reduce the yield. If the ratio is higher than 0.30, there arises problems such as corrosion of reactors and the combustion of 1,3-dialkylnaphthalene is promoted to reduce the yield.

The amount of the catalyst heavy metal to be used, in terms of the ratio of the total amount of heavy metal to the amount of solvent to be fed into the reaction zone, is preferably 0.03 to 2% by weight and more preferably 0.05 to 1% by weight. If the amount is too low, the reaction does not proceed sufficiently to make the production of reaction intermediates dominant. If the amount is too high, the combustion of 1,3-dialkylnaphthalene is promoted. The amount of the bromine compound to be used, in terms of the ratio of the total amount of bromine compound to the amount of solvent to be fed into the reaction zone, is preferably 0.01 to 2% by weight and more preferably 0.03 to 1% by weight, while selected from the above range according to the amount of 1,3-dialkylnaphthalene to be fed. The atomic ratio of the total heavy metal to the total bromine is preferably 0.2 to 10 and more preferably 0.5 to 5. The catalytic heavy metal and bromine compound may be added all at the initiation of reaction, and preferably added in portions, for example, a part thereof at the initial stage of reaction and the rest during the reaction continuously.

The 1,3-dialkylnaphthalene is oxidized in a liquid-phase with an oxygen-containing gas. Examples of the oxygen-containing gas include oxygen gas, air and mixed gas prepared by mixing oxygen or air with an inert gas such as nitrogen and argon, with air being most commonly used. Examples of oxidation reactor include an agitation tank and a bubble tower, with the agitation tank being suitable for ensuring a sufficient stirring. The reaction is suitably conducted in any of a batch manner, a semi-batch manner or a continuous manner, with the semi-batch manner and the continuous manner being more preferred. In the semi-batch manner, to complete the oxidation, it is preferred to continue the supply of the oxygen-containing gas for additional 5 to 90 min after stopping the supply of the raw material. In the continuous manner, it is preferred to connect a plurality of reactors in series to enhance the reaction yield.

The temperature for the liquid-phase oxidation is preferably 100 to 230° C. and more preferably 130 to 210° C. In the batch and semi-batch manners, the reaction temperature may be kept constant during the oxidation or may be set to a low level at the initiation of reaction and then gradually raised as the oxidation proceeds. In a multi-stage continuous manner, the reaction temperature may be different between the respective reactors within the above range. In the oxidation reaction, the oxygen-containing gas is continuously fed to the reactor and continuously discharged from the reactor after the reaction so as to regulate the reaction pressure preferably at 0.5 to 4 MPaG and more preferably at 0.7 to 3 MPaG. The oxygen-containing gas is fed into the reaction system so as to control the oxygen concentration in the exhaust gas from the reactor within the range of preferably 0.1 to 8% by volume and more preferably 0.5 to 5% by volume. If the combustion of 1,3-dialkylnaphthalene is promoted, it is preferred to reduce the oxygen concentration to 0.5 to 2% by volume.

A large amount of the solvent accompanying the exhaust gas and the water generated during the oxidation are condensed in a reflux condenser attached to the reactor. The condensed solvent and water are usually returned to the reactor, but a part thereof may be discharged out of the reaction system to control the water concentration in the reactor. The residence time of the reaction solution within the reactor is preferably 0.5 to 5 h. In case of a reactor system including a plurality of reactors connected in series, an overall residence time is preferably regulated within 0.5 to 5 h.

The reaction product solution from the liquid-phase oxidation is cooled preferably to about 10 to about 120° C., more preferably to about 20 to about 50° C. to precipitate crude 1,3-NDCA crystals. The crystallization may be performed by any of a batch manner, a semi-batch manner and a continuous manner. The crude crystals are separated from the reaction product solution by filtration or centrifugation. If required, the separated crude crystals are reslurry-washed or rinsed with water or a water-acetic acid mixture to remove organic impurities, metals, etc. contained in the crystals.

In the present invention, a reaction mother liquor obtained by solid-liquid separation of the reaction product solution from the liquid-phase oxidation may be reused in the liquid-phase oxidation. Since the reaction mother liquor contains reaction inhibitors such as high-boiling substances, preferably 90% by weight or less, more preferably 60% by weight or less of the reaction mother liquor is reused to prevent the accumulation thereof To suitably conduct the oxidation, a part of the mother liquor is preferably distilled before reuse to remove the water generated during the oxidation. The water is discharged from the top of distillation column so as to reduce the water content in the solvent to preferably 2 to 50% by weight and more preferably 5 to 30% by weight. It is industrially highly disadvantageous to reduce the water content to an extremely low level, because it is required to considerably increase the number of stages of the distillation column or discard the solvent together with water from the top thereof to reduce the water content nearer to zero.

Since the lower aliphatic carboxylic acid solvent having a water content of 2 to 50% by weight and the catalyst comprising the heavy metal and the bromine compound are used in the liquid-phase oxidation reaction, a production apparatus made of an ordinary anti-corrosive material such as SUS 304 and SUS 316, in some cases, suffers from corrosion such as pitting. To avoid such a problem, a production apparatus made of Ti or Zr which is coated with an oxide film on its inner surface is preferably used in the present invention, because the oxidation is performed without causing corrosion such as pitting. The reaction apparatus referred to herein includes reaction units such as a reactor and a stirrer which may be brought into contact with the catalyst solution and the reaction solution at 80° C. or higher, off-gas lines for oxidation reaction, cooling heat exchangers and scribers. 1,3-NDCA produced by the process of the invention may be made into a purified 1,3-naphthalenedicarboxylic diester by esterification and the subsequent crystallization or distillation each being conducted by known methods. The alcohol for the esterification may be optionally selected from methanol, ethanol, propanol, butanol, etc. The esterification may be conducted by known methods, for example, by heating 1,3-NDCA and the alcohol in the presence of an acid catalyst such as sulfuric acid, phosphoric acid and nitric acid or by using a diester of NDCA as a solvent in the presence of a heavy metal catalyst such as Mo. The resultant diester is preferably purified by crystallization from a solvent, for example, alcohols and aromatic hydrocarbons such as toluene and xylene and a subsequent distillation, or by a direct distillation without via the crystallization. The diester may be hydrolyzed into 1,3-NDCA.

As described above, 1,3-DMN is preferably used in the present invention as the starting dialkylnaphthalene for the production of 1,3-NDCA. In the process of the invention, although 1,3-DMN produced by known methods is usable, a high purity 1,3-DMN produced by a novel isomerization as will be described below is preferably used because the purification of 1,3-DMN is omitted and a high purity 1,3-NDCA is easily produced. The isomerization of DMN according to the invention comprises a step of isomerizing DMN in liquid phase in the presence of a catalyst comprising HF and BF₃ and a C₅-C₁₀ aliphatic saturated hydrocarbon having a five-membered or six-membered ring structure.

The stating DMN for the isomerization contains at least one of 1,4-DMN and 2,3-DMN. In the starting DMN, the total amount of the isomers belonging to the isomerization group C, i.e., the total mat of 1,4-DMN, 1,3-DMN and 2,3-DMN is preferably 99% by weight or more of the total DMN. The production method for the starting DMN is not particularly limited and, for example, 1,4-DMN described in U.S. Pat. No. 3,775,497 which is produced by a ring-forming dehydrogenation of 5-phenylhexene-2 is usable.

It is preferred that HF is substantially anhydrous. The molar ratio of HF to DMN (HF/DMN) is preferably 5 to 40 and more preferably 15 to 30. If less than 5, the isomerization fails to proceed efficiently. If the ratio is too large, a large reactor and a large recovering apparatus for HF are needed to deteriorate the production efficiency. The molar ratio of BF₃ to DMN (BF₃/DMN) is preferably 1.0 to 5 and more preferably 1.1 to 3. If less than 1.0, the isomer selection into 1,3-DMN is not sufficiently increased. No additional effect is obtained if the ratio exceeds the above range.

The alicyclic saturated hydrocarbon used in the present invention has a saturated five-membered or saturated six-membered ring structure in its molecule and has 5 to 10, preferably 5 to 8 carbon atoms. If the number of the carbon atoms exceeds 11, the dissolving power to DMN is undesirably lowered. Examples of the alicyclic saturated hydrocarbons include cyclopentane, methylcyclopentane, ethylcyclopentane, dimethylcyclopentane, cyclohexane, methylcyclohexane, ethylcyclohexane and dimethylcyclohexane. These alicyclic saturated hydrocarbons may be used singly or in combination of two or more.

The weight ratio of the alicyclic saturated hydrocarbon to DMN (hydrocarbon/DMN) is preferably 0.005 to 0.2 and more preferably 0.01 to 0.1. If less than 0.005, the reaction does not proceed sufficiently. Some commercially available aliphatic saturated hydrocarbons contain a small amount of the alicyclic saturated hydrocarbons usable in the invention. Therefore, the use of such commercially available aliphatic saturated hydrocarbon produces the same result as obtained by the sole use of the alicyclic saturated hydrocarbon, if used in an amount such that the alicyclic saturated hydrocarbon contained therein meets the weight ratio requirement mentioned above. For example, a commercially available n-hexane contains methylcyclopentane. If the commercially available n-hexane is added in an amount such that the weight ratio of methylcyclopentane contained therein to DMN falls within the above range, the same effect as described above is obtained.

The temperature for the isomerization is preferably −40 to 0° C. and more preferably −30 to 0° C. If higher than 0° C., considerable side reactions such as decomposition and polymerization of DMN occur. If lower than −40° C., the rate of isomerization is undesirably lowered.

As described above, when the isomerization of DMN is performed according to the process of the present invention, the aimed 1,3-DMN is produced in a high isomer selection with low costs in a short period of time while preventing the decomposition of DMN. The isomerization of the present invention is conducted in any of a batch manner, a semi-continuous manner and a continuous manner as long as an oily liquid phase containing DMN and a HF liquid phase are fully mixed under stirring.

The reaction product solution obtained after the isomerization is a HF solution of DMN•HF-BF₃ complex, which is decomposed into DMN and HF-BF₃ under heating. HF and BF₃ are separated together by vaporization, recovered and then reused. To avoid the heat-deterioration and isomerization of the product, the decomposition of the complex should be conducted as rapidly as possible. To allow the complex to be rapidly heat-decomposed, the decomposition is preferably conducted under reflux in a solvent inert to HF-BF₃, for example, saturated hydrocarbons such as heptane and aromatic hydrocarbons such as benzene.

The present invention is described in more detail below with reference to the examples. However, it should be noted that the following examples are only illustrative and not intended to limit the scope of the invention thereto.

In the following, the yield of aimed product was calculated from the results of gas-chromatographic analysis on the reaction product.

### EXAMPLE 1

1,3-DMN (purity: 99% by weight) used below was prepared by isomerizing 1,4-DMN in the same manner as in Example 7 and then removing high-boiling components by distillation. 1,4-DMN was prepared according to a known method by the alkenylation of ethylebenzene and butadiene followed by cyclization and dehydrogenation.

A 2-L titanium autoclave equipped with a gas outlet having a reflux condenser, a gas inlet and a stirrer was filled with water and air was fed under heating to 200° C. to form an oxide film over the inner wall. The autoclave thus treated was charged with 0.85 g of a 47 wt % aqueous solution of hydrogen bromide, 1.8 g of manganese acetate tetrahydrate, 1.7 g of cobalt acetate tetrahydrate, 760 g of acetic acid and 40 g of water, and the pressure and temperature were raised to 1.6 MPaG and 180° C. in a nitrogen atmosphere under stirring. Thereafter, the feeding of air into the autoclave was started while feeding 1,3-DMN at 50 g/h, a 47 wt % aqueous solution of hydrogen bromide at 0.21 g/h, manganese acetate tetrahydrate at 0.45 g/h, cobalt acetate tetrahydrate at 0.42 g/h, acetic acid at 190 g/h and water at 10 g/h. The feeding amount of air was controlled such that the oxygen concentration in off-gas was in the range of 1 to 4% at a reaction temperature of 190° C. After the reaction was conducted for one hour under the above conditions, the feeding of the raw material, catalysts and solvent was stopped while continuing the feeding of air for additional 5 min at a feeding rate half of the initial rate, thereby completing the reaction. The results of analysis of the reaction product are shown in Table 1. Then, the reaction product was cooled to 40° C. to precipitate crystals, which were then filtered. The wet cake was rinsed with acetic acid of the same amount and then heat-dried at 130° C. for 3 h in a nitrogen atmosphere. The purity of the obtained 1,3-NDCA was 99.6% by weight.

### EXAMPLE 2

The procedure of Example 1 was repeated as follows.

The autoclave was charged with 0.85 g of a 47 wt % aqueous solution of hydrogen bromide, 0.9 g of manganese acetate tetrahydrate, 0.85 g of cobalt acetate tetrahydrate, 380 g of acetic acid and 20 g of water, and the pressure and temperature were raised to 1.6 MPaG and 180° C. in a nitrogen atmosphere under stirring. Thereafter, the feeding of air into the autoclave was started while feeding 1,3-DMN at 50 g/h, a 47 wt % aqueous solution of hydrogen bromide at 0.21 g/h, manganese acetate tetrahydrate at 0.23 g/h, cobalt acetate tetrahydrate at 0.21 g/h, acetic acid at 95 g/h and water at 5 g/h. The feeding amount of air was controlled such that the oxygen concentration in off-gas was in the range of 1 to 4% at a reaction temperature of 190° C. After the reaction was conducted for one hour under the above conditions, the feeding of the raw material, catalysts and solvent was stopped while continuing the feeding of air for additional 5 min at a feeding rate half of the initial rate, thereby completing the reaction. The results of analysis of the reaction product are shown in Table 1.

### EXAMPLE 3

The procedure of Example 1 was repeated as follows.

The autoclave was charged with 1.7 g of a 47 wt % aqueous solution of hydrogen bromide, 1.8 g of manganese acetate tetrahydrate, 1.7 g of cobalt acetate tetrahydrate, 760 g of acetic acid and 40 g of water, and the pressure and temperature were raised to 1.6 MPaG and 180° C. in a nitrogen atmosphere under stirring. Thereafter, the feeding of air into the autoclave was started while feeding 1,3-DMN at 50 g/h, a 47 wt % aqueous solution of hydrogen bromide at 0.42 g/h, manganese acetate tetrahydrate at 0.45 g/h, cobalt acetate tetrahydrate at 0.42 g/h, acetic acid at 190 g(h and water at 10 g/h. The feeding amount of air was controlled such that the oxygen concentration in off-gas was in the range of 1 to 4% at a reaction temperature of 190° C. After the reaction was conducted for one hour under the above conditions, the feeding of the raw material, catalysts and solvent was stopped while continuing the feeding of air for additional 5 min at a feeding rate half of the initial rate, thereby completing the reaction. The results of analysis of the reaction product are shown in Table 1.

### COMPARATIVE EXAMPLE 1

The procedure of Example 1 was repeated as follows.

The autoclave was charged with 0.51 g of a 47 wt % aqueous solution of hydrogen bromide, 1.8 g of manganese acetate tetrahydrate, 1.7 g of cobalt acetate tetrahydrate, 760 g of acetic acid and 40 g of water, and the pressure and temperature were raised to 1.6 MPaG and 180° C. in a nitrogen atmosphere under stirring. Thereafter, the feeding of air into the autoclave was started while feeding 1,3-DMN at 50 g/h, a 47 wt % aqueous solution of hydrogen bromide at 0.13 g/h, manganese acetate tetrahydrate at 0.45 g/h, cobalt acetate tetrahydrate at 0.42 g/h, acetic acid at 190 g/h and water at 10 g/h. The feeding amount of air was controlled such that the oxygen concentration in off-gas was in the range of 1 to 4% at a reaction temperature of 190° C. After the reaction was conducted for one hour under the above conditions, the feeding of the raw material, catalysts and solvent was stopped while continuing the feeding of air for additional 5 min at a feeding rate half of the initial rate, thereby completing the reaction. The results of analysis of the reaction product are shown in Table 1.

### COMPARATIVE EXAMPLE 2

The same procedure of Example 1 was repeated except for feeding 1,3-DMN at 100 g/h for one hour. The results of analysis of the reaction product are shown in Table 1.

### COMPARATIVE EXAMPLE 3

The procedure of Example 1 was repeated as follows.

The autoclave was charged with 11.3 g of a 47 wt % aqueous solution of hydrogen bromide, 6.48 g of manganese acetate tetrahydrate, 0.68 g of cobalt acetate tetrahydrate, 760 g of acetic acid and 40 g of water, and the pressure and temperature were raised to 1.6 MPaG and 180° C. in a nitrogen atmosphere under stirring. Thereafter, the feeding of air into the autoclave was started while feeding 1,3-DMN at 50 g/h, a 47 wt % aqueous solution of hydrogen bromide at 10 g/h, manganese acetate tetrahydrate at 1.62 g/h, cobalt acetate tetrahydrate at 0.17 g/h, acetic acid at 190 g/h and water at 10 g/h. The feeding amount of air was controlled such that the oxygen concentration in off-gas was in the range of 0 to 4% at a reaction temperature of 190° C. After the reaction was conducted for one hour under the above conditions, the feeding of the raw material, catalysts and solvent was stopped while continuing the feeding of air for additional 5 min at a feeding rate half of the initial rate, thereby completing the reaction. The results of analysis of the reaction product are shown in Table 1.

TABLE 1

| | | | | | Yield (mol %) | | |
|---|---|---|---|---|---|---|---|
| Catalyst metal | | | | | | Formyl | |
| concentration*¹ (ppm) | | | | | | naphthoic | Phthalic |
| Co | Mn | Br | SR*² | Br/DMN*³ | NDCA | acid | acid |

Examples

| | | | | | | | | |
|---|---|---|---|---|---|---|---|---|
| 1 | 500 | 500 | 500 | 20 | 0.019 | 73 | 0.6 | 7 |
| 2 | 500 | 500 | 1000 | 10 | 0.019 | 69 | 0.9 | 7 |
| 3 | 500 | 500 | 1000 | 20 | 0.039 | 72 | 0.5 | 6 |

Comparative Examples

| | | | | | | | | |
|---|---|---|---|---|---|---|---|---|
| 1 | 500 | 500 | 300 | 20 | 0.012 | 38 | 8 | 7 |
| 2 | 500 | 500 | 500 | 10 | 0.010 | 35 | 9 | 7 |
| 3 | 200 | 1800 | 10000 | 20 | 0.39 | 30 | 0.1 | 5 |

*¹concentration of each metal in the solvent by weight.
*²weight ratio of the solvent to 1,3-DMN.
*³ratio of Br (including initial charge) to the fed 1,3-DMN by atoms/molecules.

### EXAMPLE 4

1,3-DMN (purity: 94% by weight) used below was prepared by isomerizing 1,4-DMN in the same manner as in Reference Example 1 and then removing high-boiling components by distillation. 1,4-DMN was prepared according to a known method by the alkenylation of ethylebenzene and butadiene followed by cyclization and dehydrogenation.

The same procedure of Example 1 was repeated except for using 1,3-DMN prepared above. The results of analysis of the reaction product are shown in Table 2. The reaction product was cooled to 40° C. to precipitate crystals, which were then filtered. The wet cake was rinsed with acetic acid of the same amount and then heat-dried at 130° C. for 3 h in a nitrogen atmosphere. The purity of the obtained 1,3-NDCA was 97.1% by weight.

### EXAMPLE 5

The same procedure of Example 2 was repeated except for using 1,3-DMN of Example 4. The results of analysis of the reaction product are shown in Table 2.

### EXAMPLE 6

The same procedure of Example 3 was repeated except for using 1,3-DMN of Example 4. The results of analysis of the reaction product are shown in Table 2.

### COMPARATIVE EXAMPLE 4

The same procedure of Comparative Example 1 was repeated except for using 1,3-DMN of Example 4. The results of analysis of the reaction product are shown in Table 2.

### COMPARATIVE EXAMPLE 5

The same procedure of Comparative Example 2 was repeated except for using 1,3-DMN of Example 4. The results of analysis of the reaction product are shown in Table 2.

### COMPARATIVE EXAMPLE 6

The same procedure of Comparative Example 3 was repeated except for using 2,6-DMN (purity: 99.0% by weight) to produce 2,6-NDCA. The results of analysis of the reaction product are shown in Table 2.

TABLE 2

| | | | | | Yield (mol %) | | |
|---|---|---|---|---|---|---|---|
| Catalyst metal | | | | | | Formyl | |
| concentration*¹ (ppm) | | | | | | naphthoic | Phthalic |
| Co | Mn | Br | SR*² | Br/DMN*³ | NDCA | acid | acid |

Examples

| | | | | | | | | |
|---|---|---|---|---|---|---|---|---|
| 4 | 500 | 500 | 500 | 20 | 0.019 | 72 | 0.7 | 7 |
| 5 | 500 | 500 | 1000 | 10 | 0.019 | 68 | 0.9 | 8 |
| 6 | 500 | 500 | 1000 | 20 | 0.039 | 72 | 0.6 | 6 |

Comparative Examples

| | | | | | | | | |
|---|---|---|---|---|---|---|---|---|
| 4 | 500 | 500 | 300 | 20 | 0.012 | 36 | 9 | 8 |
| 5 | 500 | 500 | 500 | 10 | 0.010 | 34 | 9 | 7 |
| 6 | 200 | 1800 | 10000 | 20 | 0.39 | 79 | 0.1 | 0.1 |
| | | | | | | (2,6-) | | |

*¹concentration of each metal in the solvent by weight.
*²weight ratio of the solvent to 1,3-DMN or 2,6-DMN.
*³ratio of Br (including initial charge) to the fed 1,3-DMN or 2,6-DMN by atoms/molecules.

The production of 1,3-DMN by the isomerization process of the invention will be described below. The starting 1,4-DMN (99.0% purity as measured by a gas-chromatographic analysis using a nonpolar column) used herein was prepared in accordance with a known method by the alkenylation of ethylbenzene and 1,3-butadiene followed by cyclization and then dehydrogenation.

### EXAMPLE 7

A 500-mL temperature-controllable autoclave (SUS316L) equipped with an electromagnetic stirrer was charged with 150 g (7.5 mol) of anhydrous HF and 22 g (0.32 mol) of BF₃. After cooling the liquid contents to −10° C., 39 g (0.25 mol) of 1,4-DMN containing 0.8 g of methylcyclopentane was added while stirring the contents. After holding the temperature at −10° C. for 60 min, the contents were poured into ice, diluted with hexane and then neutralized to obtain an oil phase, which was analyzed by gas chromatography to determine the ratio of DMN isomers. The results are shown in Table 3.

### EXAMPLE 8

The isomerization and treatment of the reaction product solution were conducted in the same manner as in Example 7 except for using 0.8 g of cyclohexane instead of methylcyclopentane. The results are shown in Table 3.

### EXAMPLE 9

The isomerization and treatment of the reaction product solution were conducted in the same manner as in Example 7 except for using 0.8 g of methylcyclohexane instead of methylcyclopentane. The results are shown in Table 3.

### EXAMPLE 10

The isomerization and treatment of the reaction product solution were conducted in the same manner as in Example 7 except for using 78 g of n-hexane containing 1% by weight of methylcyclopentane in place of the sole use of methylcyclopentane. The results are shown in Table 3.

### REFERENCE EXAMPLE 1

The isomerization and treatment of the reaction product solution were conducted in the same manner as in Example 7 except for omitting the addition of methylcyclopentane. The results are shown in Table 3. The ratio of 1,3-DMN was low as compared to Example 1.

### REFERENCE EXAMPLE 2

The isomerization and treatment of the reaction product solution were conducted in the same manner as in Example 7 except for adding 0.8 g of n-heptane, a non-alicyclic saturated hydrocarbon, to 1,4-DMN in place of methylcyclopentane. The results are shown in Table 3. The ratio of 1,3-DMN was low as compared to Example 1.

TABLE 3

| | | | | | Reference | |
|---|---|---|---|---|---|---|
| | Examples | | | | Examples | |
| | 7 | 8 | 9 | 10 | 1 | 2 |
| Hydrocarbon | | | | | | |
| kinds | methyl- | cyclohexane | methyl- | n-hexane/ | — | n-heptane |
| | cyclopentane | | cyclohexane | methyl- | | |
| | | | | cyclopentane | | |
| hydrocarbon/DMN (by weight) | 0.02 | 0.02 | 0.02 | 2.0/0.02 | — | 0.02 |
| Composition of product (wt %) | | | | | | |
| low boiling component*⁴ | 0.3 | 0.2 | 0.3 | 0.6 | 0.7 | 0.1 |
| high boiling component*⁵ | 2.2 | 2.3 | 2.2 | 0.8 | 1.0 | 1.0 |
| total DMN | 97.5 | 97.5 | 97.4 | 98.6 | 98.3 | 98.9 |
| Ratio of DMN isomers (wt %) | | | | | | |
| 1,3-isomer | 99.8 | 99.0 | 98.7 | 99.4 | 93.6 | 92.8 |
| 1,4-isomer | 0.2 | 1.0 | 1.3 | 0.6 | 6.4 | 7.2 |
| 2,3-isomer | 0 | 0 | 0 | 0 | 0 | 0 |

*⁴reaction product having a boiling point lower than that of produced DMN.
*⁵reaction product having a boiling point higher than that of produced DMN.

In Reference Example 1 where no alicyclic saturated hydrocarbon was added to DMN and Reference Example 2 where n-heptane was added to DMN in place of the alicyclic saturated hydrocarbon, the ratio of 1,3-DMN was as low as about 93% to show that the isomerization into 1,3-isomer was not sufficiently completed. In contrast, in the examples of the invention where the alicyclic saturated hydrocarbon was added, the ratio of 1,3-DMN was as high as about 99% to show that the isomerization was completed with a high isomer selection into 1,3-DMN. In Example 7 where n-hexane containing 1% by weight of methylcyclopentane was used, the same ratio of 1,3-DMN as in Example 1 where methylcyclopentane was singly used was achieved.

As described above, 1,3-NDCA is efficiently produced by the process of the invention with low costs. 1,3-NDCA and esters thereof are extensively used in wide applications such as raw materials for polyester resins and fibers having unprecedentedly useful functions, raw materials for liquid crystal polymers, modifiers for polyesters, curing agents for epoxy resins, raw materials for medicines or agricultural chemicals, and raw materials for lubricants.

In accordance with the process of the invention for isomerizing a mixture of 1,4-isomer, 1,3-isomer and 2,3-isomer which belong to the isomerization group C, 1,4-isomer and 2,3-isomer are efficiently isomerized into 1,3-isomer at high isomer selections in a short period of time. By using 1,3-DMN produced by the process of the invention as the starting 1,3-dialkylnaphthalene in the process of the invention for producing 1,3-NDCA, a highly pure 1,3-NDCA is produced more efficiently.

1. A process for producing 1,3-naphthalenedicarboxylic acid, comprising a step of subjecting 1,3-dialkylnaphthalene to a liquid-phase oxidation with an oxygen-containing gas in the presence of a C₂-C₆ lower aliphatic carboxylic acid solvent and a catalyst comprising a heavy metal and a bromine compound, wherein a ratio of the total number of bromine atoms fed into a reaction system to the total number of 1,3-dialkylnaphthalene molecules fed into the reaction system is regulated within a range of 0.015 to 0.30.

2. The process according to claim 1, wherein the 1,3-dialkylnaphthalene is 1,3-dimethylnaphthalene.

3. The process according to claim 2, wherein the 1,3-dimethylnaphthalene has a purity of 97% by weight or higher.

4. The process according to claim 1, wherein the bromine compound is used in an amount of 0.01 to 2% by weight of the solvent in terms of bromine atom, the heavy metal is used in an amount of 0.03 to 2% by weight of the solvent in terms of heavy metal atom, and an atomic ratio of heavy metal to bromine is 0.2 to 10.

5. The process according to claim 1, wherein a water content of the lower aliphatic carboxylic acid solvent is 2 to 50% by weight.

6. The process according to claim 1, wherein a reaction product solution of the liquid-phase oxidation is subjected to a solid-liquid separation to precipitate and separate 1,3-naphthalenedicarboxylic acid.

7. The process according to claim 6, wherein a mother liquor separated by the solid-liquid separation is reused in the liquid-phase oxidation.

8. The process according to claim 1, wherein the liquid-phase oxidation is performed in a reaction apparatus made of Ti or Zr which is coated with an oxide film on its inner surface.

9. The process according to claim 2, wherein 1,3-dimethylnaphthalene is produced by isomerizing dimethylnaphthalene in a liquid phase in the presence of a catalyst comprising HF and BF₃ and a C₅-C₁₀ alicyclic saturated hydrocarbon having a five-membered or six-membered ring structure.

10. The process according to claim 9, wherein a weight ratio of the alicyclic saturated hydrocarbon to dimethylnaphthalene is in a range of 0.005 to 0.2.

11. The process according to claim 9, wherein the isomerization reaction is performed at −40 to 0° C.

12. The process according to claim 9, wherein dimethylnaphthalene contains at least one of 1,4-dimethylnaphthalene and 2,3-dimethylnaphthalene.

13. 1,3-Naphthalenedicarboxylic acid produced by the process as defined in claim 1.

14. A 1,3-naphthalenedicarboxylic diester produced by esterifying 1,3-naphthalenedicarboxylic acid as defined in claim 13.

15. A process for producing 1,3-dimethylnaphthalene, which comprises a step of isomerizing dimethylnaphthalene in a liquid phase in the presence of a catalyst comprising HF and BF₃ and a C₅-C₁₀ alicyclic saturated hydrocarbon having a five-membered or six-membered ring structure.

16. The process according to claim 15, wherein a weight ratio of the alicyclic saturated hydrocarbon to dimethylnaphthalene is in a range of 0.005 to 0.2.

17. The process according to claim 15, wherein the isomerization reaction is performed at −40 to 0° C.

18. The process according to claim 15, wherein dimethylnaphthalene contains at least one of 1,4-dimethylnaphthalene and 2,3-dimethylnaphthalene.
